(19) United States
(12) Patent Application Publication
(10) Pub. No.: US 2021/0154420 A1
(43) Pub. Date: May 27, 2021

(54) VAPORIZER DEVICE DOSE CONSUMPTION CONFIGURATIONS

(71) Applicant: JUUL Labs, Inc., San Francisco, CA (US)

(72) Inventors: Erik Jorgensen, San Francisco, CA (US); Katherine Murphy, San Francisco, CA (US); Colt Stander, Millbrae, CA (US); Matthew Czapar, San Francisco, CA (US); Oliver Farshi, Oakland, CA (US)

(21) Appl. No.: 16/953,081

(22) Filed: Nov. 19, 2020

Related U.S. Application Data
(60) Provisional application No. 63/014,479, filed on Apr. 23, 2020.
(60) Provisional application No. 62/938,893, filed on Nov. 21, 2019.

(51) Int. Cl. A61M 15/06; A61M 15/00

(52) U.S. Cl. CPC A61M 15/06; A61M 15/0071; A61M 15/0083

(57) ABSTRACT

Features relating to vaporizer device configurations related to dose consumption are provided. Aspects relate to configuring a vaporizer device with respect to providing feedback to a user with respect to dose consumption of one or more vaporizable materials being vaporized and inhaled by a user of a vaporizer device. The vaporizer device monitors dose consumption, based on at least applied energy amounts, and generates feedback in the form of a haptics pulse, an audio indication, a visual indication, or a combination thereof. A timer is used to determine if a dose falls within a series of doses or is part of a new series. A timeout period is used to stop or limit use following the completion of a series of doses. Data relating to the dose consumption and the series of doses may be stored on and associated with a cartridge, allowing for resumption of the dose consumption aspects.

### CROSS-REFERENCE TO RELATED APPLICATIONS

This application claims priority to U.S. Provisional Patent Application No. 62/938,893, filed on Nov. 21, 2019, and U.S. Provisional Patent Application No. 63/014,479, filed on Apr. 23, 2020, the contents of which are herein incorporated by reference in their entirety.

### TECHNICAL FIELD

The current subject matter described herein relates generally to vaporizer devices, such as portable, personal vaporizer devices for generating and delivering an inhalable aerosol from one or more vaporizable materials, and more particularly relates to vaporizer device configurations.

### BACKGROUND

Vaporizing devices, including electronic vaporizers or e-vaporizer devices, allow the delivery of vapor and aerosol containing one or more active ingredients by inhalation of the vapor and aerosol. Electronic vaporizer devices are gaining increasing popularity both for prescriptive medical use, in delivering medicaments, and for consumption of nicotine, tobacco, other liquid-based substances, and other plant-based smokeable materials, such as cannabis, including solid (e.g., loose-leaf or flower) materials, solid/liquid (e.g., suspensions, liquid-coated) materials, wax extracts, and prefilled pods (cartridges, wrapped containers, etc.) of such materials. Electronic vaporizer devices in particular may be portable, self-contained, and convenient for use.

### SUMMARY

Aspects of the current subject matter relate to providing feedback to a user with respect to dose consumption of one or more vaporizable materials being vaporized and inhaled by a user of a vaporizer device.

According to an aspect of the current subject matter, a method includes, responsive to a user inhale on a vaporizer device, initiating a timer to a predefined timer value and applying energy to a heating element of the vaporizer device; determining consumption of a first dose of vaporizable material vaporized by the heating element, the determination of the consumption of the first dose based at least on an amount of the energy applied to the heating element; outputting, in response to a determination by the vaporizer device that one or more doses out of a predefined number of doses remain, a first feedback; responsive to a subsequent user inhale on the vaporizer device, re-initiating the timer to the predefined timer value and applying energy to the heating element; determining consumption of a second dose of vaporizable material vaporized by the heating element, the determination of the consumption of the second dose based at least on the amount of the energy applied to the heating element; and outputting a second feedback.

According to an inter-related aspect, a vaporizer device includes at least one data processor and at least one memory storing instructions which, when executed by the at least one data processor, cause operations including, responsive to a user inhale on the vaporizer device, initiate a timer to a predefined timer value and apply energy to a heating element of the vaporizer device; determine consumption of a first dose of vaporizable material vaporized by the heating element, the determination of the consumption of the first dose based at least on an amount of the energy applied to the heating element; output, in response to a determination that one or more doses out of a predefined number of doses remain, a first feedback; responsive to a subsequent user inhale on the vaporizer device, re-initiate the timer to the predefined timer value and apply energy to the heating element; determine consumption of a second dose of vaporizable material vaporized by the heating element, the determination of the consumption of the second dose based at least on the amount of the energy applied to the heating element; and output a second feedback.

According to an inter-related aspect, a non-transitory computer readable medium is provided, the non-transitory computer readable medium storing instructions, which when executed by at least one data processor, result in operations including, responsive to a user inhale on a vaporizer device, initiating a timer to a predefined timer value and applying energy to a heating element of the vaporizer device; determining consumption of a first dose of vaporizable material vaporized by the heating element, the determination of the consumption of the first dose based at least on an amount of the energy applied to the heating element; outputting, in response to a determination by the vaporizer device that one or more doses out of a predefined number of doses remain, a first feedback; responsive to a subsequent user inhale on the vaporizer device, re-initiating the timer to the predefined timer value and applying energy to the heating element; determining consumption of a second dose of vaporizable material vaporized by the heating element, the determination of the consumption of the second dose based at least on the amount of the energy applied to the heating element; and outputting a second feedback.

In some variations, one or more of the features disclosed herein including the following features can optionally be included in any feasible combination. In response to the determination of the consumption of the first dose, a dose counter may be incremented, where a value of the dose counter reflects a number of doses consumed. Responsive to the subsequent user inhale, a determination may be made that the period of time between the user inhale and subsequent user inhale exceeds the predefined timer value, and the dose counter may be cleared. A determination, in response to the determination of the consumption of the second dose, may be made that the predetermined number of doses are consumed, and a timeout period may be entered in response to the determination that the predetermined number of doses are consumed, where during the timeout period, the heating element does not respond to activation commands. An override command during the timeout period may be received, and responsive to the override command, the timer may be re-initiated to the predefined timer value and energy may be applied to the heating element. The second feedback may be indicative of the predetermined number of doses being consumed. The first feedback may be representative of the consumption of the first dose and the second feedback may be representative of the consumption of the second dose. The determination of the consumption of the first dose may be based on the amount of the energy applied to the heating element equal to or exceeding a predetermined value, the predetermined value indicative of at least a partial consumption of the vaporizable material. Storage on a data tag of a cartridge in use with the vaporizer device may include storing one or more of the predefined timer value, the amount of the energy applied to the heating element, and the predefined number of doses.

The details of one or more variations of the subject matter described herein are set forth in the accompanying drawings and the description below. Other features and advantages of the subject matter described herein will be apparent from the description and drawings, and from the claims. The claims that follow this disclosure are intended to define the scope of the protected subject matter.

### DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings:

FIG. 1A-FIG. 1F illustrate features of a vaporizer device including a vaporizer body and a cartridge consistent with implementations of the current subject matter;

FIG. 2 is a schematic block diagram illustrating features of a vaporizer device having a cartridge and a vaporizer body consistent with implementations of the current subject matter;

FIG. 3 illustrates communication between a vaporizer device, a user device, and a server consistent with implementations of the current subject matter;

FIG. 4A-FIG. 4C illustrate timing and dose feedback aspects of a vaporizer device consistent with implementations of the current subject matter;

FIG. 5 depicts a chart illustrating features of a process consistent with implementations of the current subject matter;

FIG. 6 depicts a swim lane diagram illustrating operations of a user, a user device, and a vaporizer device consistent with implementations of the current subject matter;

FIG. 7 depicts a chart illustrating features of a process consistent with implementations of the current subject matter; and

FIG. 8 depicts a chart illustrating features of another process consistent with implementations of the current subject matter.

When practical, similar reference numbers denote similar structures, features, or elements.

### DETAILED DESCRIPTION

Aspects of the current subject matter relate to vaporizer device configurations including controlling operation of a vaporizer device with respect to dose consumption of one or more doses of one or more vaporizable materials being vaporized and inhaled by a user of the vaporizer device.

Implementations of the current subject matter refer to doses of aerosol for consumption. According to aspects of the current subject matter, a dose is defined as a fixed amount of aerosol generated by the vaporizer device for consumption by the user as a number of puffs taken by the user until the fixed amount of aerosol is consumed or inhaled. In some implementations, a dose may also be referred to as a session; and the terms dose and session may be used interchangeably herein. In some instances, a dose is fixed or selected by an entity, such as a manufacturer or distributor of the cartridge or the vaporizer device, or a care giver, medical facility, and/or the like for controlling consumption by the user. In some instances, the dose is user adjustable and/or user configurable. In some instances, the dose is represented by a dose size and/or a dose size setting. The dose may be correlated with amount of energy supplied to a heating element of the cartridge, as further described herein.

Before providing additional details regarding aspects of vaporizer device configurations, the following provides a description of some examples of vaporizer devices including a vaporizer body and a cartridge. The following descriptions are meant to be exemplary, and aspects related to vaporizer device configurations consistent with the current subject matter are not limited to the example vaporizer devices described herein.

Implementations of the current subject matter include devices relating to vaporizing of one or more materials for inhalation by a user. The term “vaporizer” may be used generically in the following description and may refer to a vaporizer device, such as an electronic vaporizer. Vaporizers consistent with the current subject matter may be referred to by various terms such as inhalable aerosol devices, aerosolizers, vaporization devices, electronic vaping devices, electronic vaporizers, vape pens, etc. Examples of vaporizers consistent with implementations of the current subject matter include electronic vaporizers, electronic cigarettes, e-cigarettes, or the like. In general, such vaporizers are often portable, hand-held devices that heat a vaporizable material to provide an inhalable dose of the material. The vaporizer may include a heater configured to heat a vaporizable material which results in the production of one or more gas-phase components of the vaporizable material. A vaporizable material may include liquid and/or oil-type plant materials, or a semi-solid like a wax, or plant material such as leaves or flowers, either raw or processed. The gas-phase components of the vaporizable material may condense after being vaporized such that an aerosol is formed in a flowing air stream that is deliverable for inhalation by a user. The vaporizers may, in some implementations of the current subject matter, be particularly adapted for use with an oil-based vaporizable material, such as cannabis-derived oils although other types of vaporizable materials may be used as well.

One or more features of the current subject matter, including one or more of a cartridge (also referred to as a vaporizer cartridge or pod) and a reusable vaporizer device body (also referred to as a vaporizer device base, a body, a vaporizer body, or a base), may be employed with a suitable vaporizable material (where suitable refers in this context to being usable with a device whose properties, settings, etc. are configured or configurable to be compatible for use with the vaporizable material). The vaporizable material may include one or more liquids, such as oils, extracts, aqueous or other solutions, etc., of one or more substances that may be desirably provided in the form of an inhalable aerosol. The cartridge may be inserted into the vaporizer body, and then the vaporizable material heated which results in the inhalable aerosol.

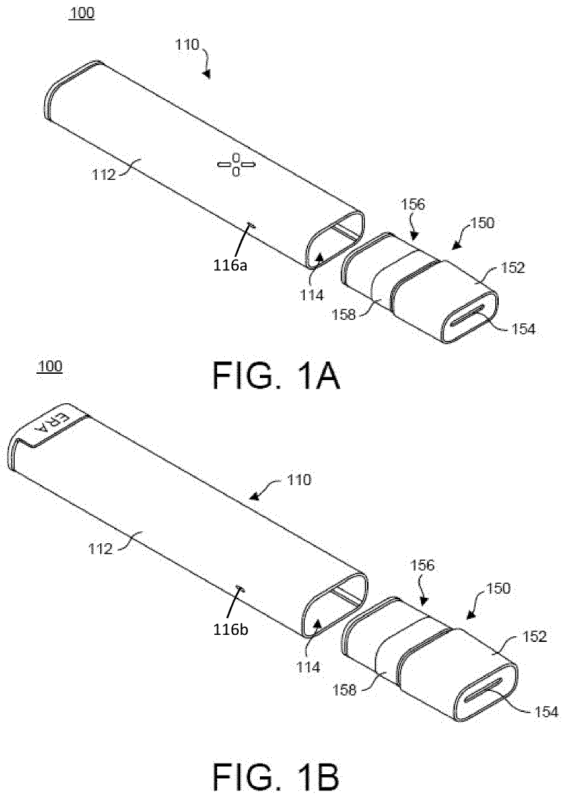

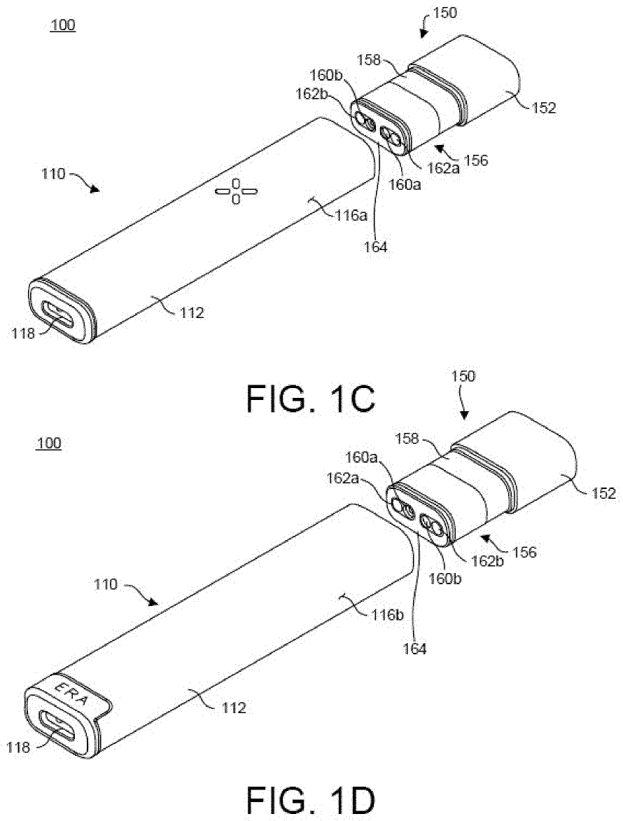

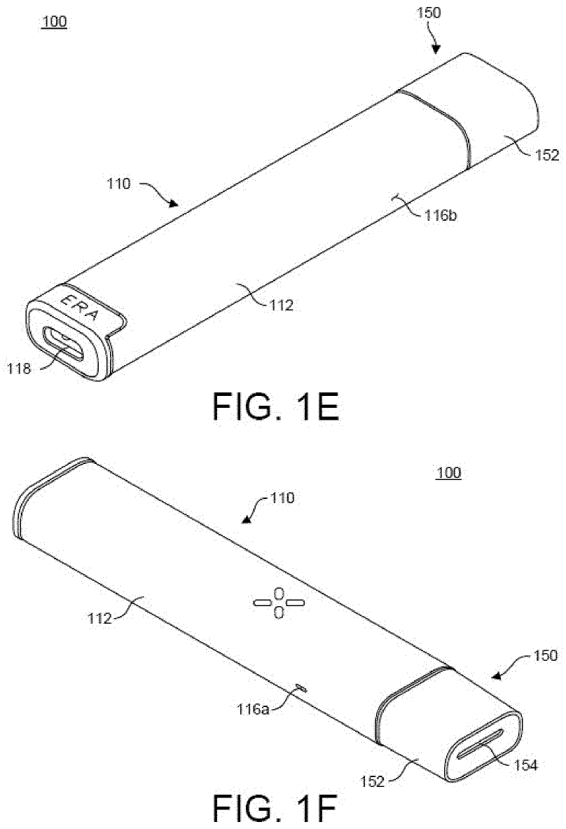

FIG. 1A-FIG. 1F illustrates features of a vaporizer device 100 including a vaporizer body 110 and a cartridge 150 consistent with implementations of the current subject matter. FIG. 1A is a bottom perspective view, and FIG. 1B is a top perspective view of the vaporizer device 100 with the cartridge 150 separated from a cartridge receptacle 114 on the vaporizer body 110. Both of the views in FIG. 1A and FIG. 1B are shown looking towards a mouthpiece 152 of the cartridge 150. FIG. 1C is a bottom perspective view, and FIG. 1D is a top perspective view of the vaporizer device with the cartridge 150 separated from the cartridge receptacle 114 of the vaporizer body 110. FIG. 1C and FIG. 1D are shown looking toward the distal end of the vaporizer body 110. FIG. 1E is top perspective view, and FIG. 1F is a bottom perspective view of the vaporizer device 100 with the cartridge 150 engaged for use with the vaporizer body 110.

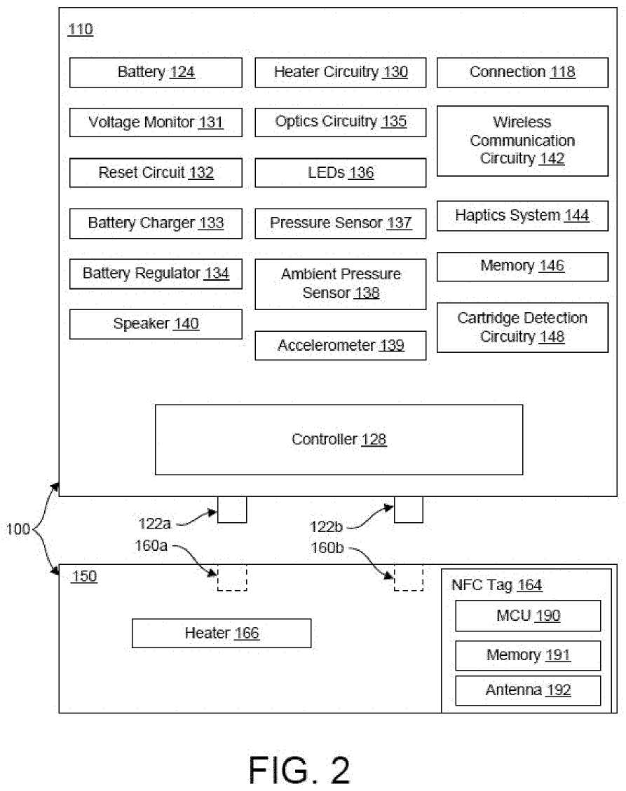

As shown in FIG. 1A-FIG. 1D, the cartridge 150 includes, at the proximal end, a mouthpiece 152 that is attached over a cartridge body 156 that forms a reservoir or tank 158 that holds a vaporizable material. The cartridge body 156 may be transparent, translucent, opaque, or a combination thereof. The mouthpiece 152 may include one or more openings 154 (see FIG. 1A, FIG. 1B, FIG. 1F) at the proximal end out of which vapor may be inhaled, by drawing breath through the vaporizer device 100. The distal end of the cartridge body 156 may couple to and be secured to the vaporizer body 110 within the cartridge receptacle 114 of the vaporizer body 110. Power pin receptacles 160a,b (see FIG. 1C, FIG. 1D) of the cartridge 150 mate with respective power pins or contacts 122a,b (see, for example, FIG. 2) of the vaporizer body 110 that extend into the cartridge receptacle 114. The cartridge 150 also includes air flow inlets 162a,b on the distal end of the cartridge body 156.

A tag 164, such as a data tag, a near-field communication (NFC) tag, or other type of wireless transceiver or communication tag, may be positioned on at least a portion of the distal end of the cartridge body 156. As shown in FIG. 1C and FIG. 1D, the tag 164 may substantially surround the power pin receptacles 160a,b and the air flow inlets 162a,b, although other configurations of the tag 164 may be implemented as well. For example, the tag 164 may be positioned between the power pin receptacle 160a and the power pin receptacle 160b, or the tag 164 may be shaped as a circle, partial circle, oval, partial oval, or any polygonal shape encircling or partially encircling the power pin receptacles 160a,b and the air flow inlets 162a,b or a portion thereof.

In the example of FIG. 1A, the vaporizer body 110 has an outer shell or cover 112 that may be made of various types of materials, including for example aluminum (e.g., AL6063), stainless steel, glass, ceramic, titanium, plastic (e.g., Acrylonitrile Butadiene Styrene (ABS), Nylon, Polycarbonate (PC), Polyethersulfone (PESU), and the like), fiberglass, carbon fiber, and any hard, durable material. The proximal end of the vaporizer body 110 includes an opening forming the cartridge receptacle 114, and the distal end of the vaporizer body 110 includes a connection 118, such as, for example, a universal serial bus Type C (USB-C) connection and/or the like. The cartridge receptacle 114 portion of the vaporizer body 110 includes one or more openings (air inlets) 116a,b that extend through the outer shell 112 to allow airflow therein, as described in more detail below. The vaporizer body 110 as shown has an elongated, flattened tubular shape that is curvature-continuous, although the vaporizer body 110 is not limited to such a shape. The vaporizer body 110 may take the form of other shapes, such as, for example, a rectangular box, a cylinder, and the like.

The cartridge 150 may fit within the cartridge receptacle 114 by a friction fit, snap fit, and/or other types of secure connection. The cartridge 150 may have a rim, ridge, protrusion, and/or the like for engaging a complimentary portion of the vaporizer body 110. While fitted within the cartridge receptacle 114, the cartridge 150 may be held securely within but still allow for being easily withdrawn to remove the cartridge 150.

Although FIG. 1A-FIG. 1F illustrate a certain configuration of the vaporizer device 100, the vaporizer device 100 may take other configurations as well.

FIG. 2 is a schematic block diagram illustrating components of the vaporizer device 100 having the cartridge 150 and the vaporizer body 110 consistent with implementations of the current subject matter. Included in the vaporizer body 110 is a controller 128 that includes at least one processor and/or at least one memory configured to control and manage various operations among the components of the vaporizer device 100 described herein.

Heater control circuitry 130 of the vaporizer body 110 controls a heater 166 of the cartridge 150. The heater 166 may generate heat to provide vaporization of the vaporizable material. For example, the heater 166 may include a heating coil (e.g., a resistive heater) in thermal contact with a wick which absorbs the vaporizable material, as described in further detail below.

A battery 124 is included in the vaporizer body 110, and the controller 128 may control and/or communicate with a voltage monitor 131 which includes circuitry configured to monitor the battery voltage, a reset circuit 132 configured to reset (e.g., shut down the vaporizer device 100 and/or restart the vaporizer device 100 in a certain state), a battery charger 133, and a battery regulator 134 (which may regulate the battery output, regulate charging/discharging of the battery, and provide alerts to indicate when the battery charge is low, etc.).

The power pins 122a,b of the vaporizer body 110 engage the complementary power pin receptacles 160a,b of the cartridge 150 when the cartridge 150 is engaged with the vaporizer body 110. Alternatively, power pins may be part of the cartridge 150 for engaging complementary power pin receptacles of the vaporizer body 110. The engagement allows for the transfer of energy from an internal power source (e.g., the battery 124) to the heater 166 in the cartridge 150. The controller 128 may regulate the power flow (e.g., an amount or current and/or a voltage amount) to control a temperature at which the heater 166 heats the vaporizable material contained in the reservoir 158. According to implementations of the current subject matter, a variety of electrical connectors other than a pogo-pin and complementary pin receptacle configuration may be used to electrically connect the vaporizer body 110 and the cartridge 150, such as for example, a plug and socket connector.

The controller 128 may control and/or communicate with optics circuitry 135 (which controls and/or communicates with one or more displays such as LEDs 136 which may provide user interface output indications), a pressure sensor 137, an ambient pressure sensor 138, an accelerometer 139, and/or a speaker 140 configured to generate sound or other feedback to a user.

The pressure sensor 137 may be configured to sense a user drawing (i.e., inhaling) on the mouthpiece 152 and activate the heater control circuitry 130 of the vaporizer body 110 to accordingly control the heater 166 of the cartridge 150. In this way, the amount of current supplied to the heater 166 may be varied according the user's draw (e.g., additional current may be supplied during a draw, but reduced when there is not a draw taking place). The ambient pressure sensor 138 may be included for atmospheric reference to reduce sensitivity to ambient pressure changes and may be utilized to reduce false positives potentially detected by the pressure sensor 137 when measuring draws from the mouthpiece 152.

The accelerometer 139 (and/or other motion sensors, capacitive sensors, flow sensors, strain gauge(s), or the like) may be used to detect user handling and interaction, for example, to detect movement of the vaporizer body 110 (such as, for example, tapping, rolling, and/or any other deliberate movement associated with the vaporizer body 110).

The vaporizer body 110, as shown in FIG. 2, includes wireless communication circuitry 142 that is connected to and/or controlled by the controller 128. The wireless communication circuitry 142 may include a near-field communication (NFC) antenna that is configured to read from and/or write to the tag 164 of the cartridge 150. Alternatively or additionally, the wireless communication circuitry 142 may be configured to automatically detect the cartridge 150 as it is being inserted into the vaporizer body 110. In some implementations, data exchanges between the vaporizer body 110 and the cartridge 150 take place over NFC. In some implementations, data exchanges between the vaporizer body 110 and the cartridge 150 may take place via a wired connection such as various wired data protocols.

The wireless communication circuitry 142 may include additional components including circuitry for other communication technology modes, such as Bluetooth circuitry, Bluetooth Low Energy circuitry, Wi-Fi circuitry, cellular (e.g., LTE, 4G, and/or 5G) circuitry, and associated circuitry (e.g., control circuitry), for communication with other devices. For example, the vaporizer body 110 may be configured to wirelessly communicate with a remote processor (e.g., a smartphone, a tablet, a computer, wearable electronics, a cloud server, and/or processor based devices) through the wireless communication circuitry 142, and the vaporizer body 110 may through this communication receive information including control information (e.g., for setting temperature, resetting a dose counter, etc.) from and/or transmit output information (e.g., dose information, operational information, error information, temperature setting information, charge/battery information, etc.) to one or more of the remote processors.

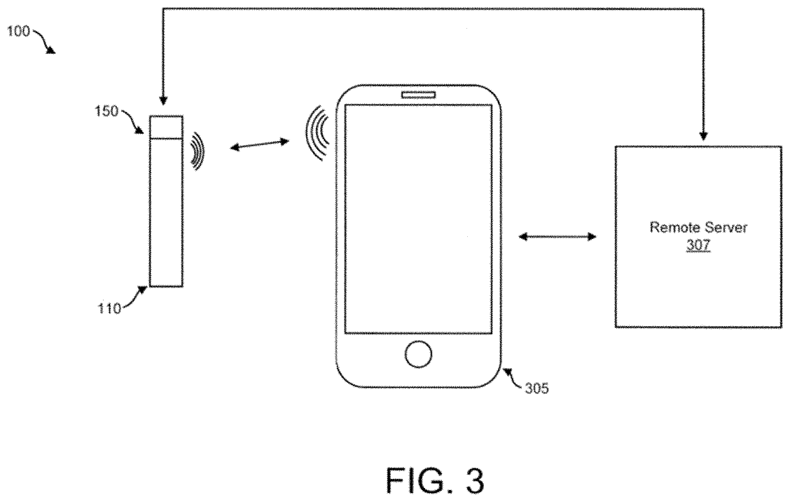

The tag 164 may be a type of wireless transceiver and may include a microcontroller unit (MCU) 190, a memory 191, and an antenna 192 (e.g., an NFC antenna) to perform the various functionalities described below with further reference to FIG. 3. NFC tag 164 may be, for example, a 1 Kbit or a 2 Kbit tag that is of type ISO/IEC 15693. NFC tags with other specifications may also be used. The tag 164 may be implemented as active NFC, enabling reading and/or writing information via NFC with other NFC compatible devices including a remote processor, another vaporizer device, and/or wireless communication circuitry 142. Alternatively, the tag 164 may be implemented using passive NFC technology, in which case other NFC compatible devices (e.g., a remote processor, another vaporizer device, and/or wireless communication circuitry 142) may only be able to read information from the tag 164.

The vaporizer body 110 may include a haptics system 144, such as an actuator, a linear resonant actuator (LRA), an eccentric rotating mass (ERM) motor, or the like that provide haptic feedback such as a vibration as a “find my device” feature or as a control or other type of user feedback signal. For example, using an app running on a user device (such as, for example, a user device 305 shown in FIG. 3), a user may indicate that he/she cannot locate his/her vaporizer device 100. Through communication via the wireless communication circuitry 142, the controller 128 sends a signal to the haptics system 144, instructing the haptics system 144 to provide haptic feedback (e.g., a vibration). The controller 128 may additionally or alternatively provide a signal to the speaker 140 to emit a sound or series of sounds. The haptics system 144 and/or speaker 140 may also provide control and usage feedback to the user of the vaporizer device 100; for example, providing haptic and/or audio feedback when a particular amount of a vaporizable material has been used or when a period of time since last use has elapsed. Alternatively or additionally, haptic and/or audio feedback may be provided as a user cycles through various settings of the vaporizer device 100. Alternatively or additionally, the haptics system 144 and/or speaker 140 may signal when a certain amount of battery power is left (e.g., a low battery warning and recharge needed warning) and/or when a certain amount of vaporizable material remains (e.g., a low vaporizable material warning and/or time to replace the cartridge 150). Alternatively or additionally, the haptics system 144 and/or speaker 140 may also provide usage feedback and/or control of the configuration of the vaporizer device 100 (e.g., allowing the change of a configuration, such as target heating rate, heating rate, etc.).

The vaporizer body 110 may include circuitry for sensing/detecting when a cartridge 150 is connected and/or removed from the vaporizer body 110. For example, cartridge-detection circuitry 148 may determine when the cartridge 150 is connected to the vaporizer body 110 based on an electrical state of the power pins 122a,b within the cartridge receptacle 114. For example, when the cartridge 150 is present, there may be a certain voltage, current, and/or resistance associated with the power pins 122a,b, when compared to when the cartridge 150 is not present. Alternatively or additionally, the tag 164 may also be used to detect when the cartridge 150 is connected to the vaporizer body 110.

The vaporizer body 110 also includes the connection (e.g., USB-C connection, micro-USB connection, and/or other types of connectors) 118 for coupling the vaporizer body 110 to a charger to enable charging the internal battery 124. Alternatively or additionally, electrical inductive charging (also referred to as wireless charging) may be used, in which case the vaporizer body 110 would include inductive charging circuitry to enable charging. The connection 118 at FIG. 2 may also be used for a data connection between a computing device and the controller 128, which may facilitate development activities such as, for example, programming and debugging, for example.

The vaporizer body 110 may also include a memory 146 that is part of the controller 128 or is in communication with the controller 128. The memory 146 may include volatile and/or non-volatile memory or provide data storage. In some implementations, the memory 146 may include 8 Mbit of flash memory, although the memory is not limited to this and other types of memory may be implemented as well.

FIG. 3 illustrates communication between the vaporizer device 100 (including the vaporizer body 110 and the cartridge 150), the user device 305 (e.g., a smartphone, tablet, laptop, and/or the like), and a remote server 307 (e.g., a server coupled to a network, a cloud server coupled to the Internet, and/or the like) consistent with implementations of the current subject matter. The user device 305 wirelessly communicates with the vaporizer device 100. A remote server 307 may communicate directly with the vaporizer device 100 or through the user device 305. The vaporizer body 110 may communicate with the user device 305 and/or the remote server 307 through the wireless communication circuitry 142. In some implementations, the cartridge 150 may establish through the tag 164 communication with the vaporizer body 110, the user device 305, and/or the remote server 307.

An application software (“app”) running on at least one of the remote processors (the user device 305 and/or the remote server 307) may be configured to control operational aspects of the vaporizer device 100 and receive information relating to operation of the vaporizer device 100. For example, the app may provide a user with capabilities to input or set desired properties or effects, such as, for example, a particular temperature or desired dose, which is then communicated to the controller 128 of the vaporizer body 110 through the wireless communication circuitry 142. The app may also provide a user with functionality to select one or more sets of suggested properties or effects that may be based on the particular type of vaporizable material in the cartridge 150. For example, the app may allow adjusting heating based on the type of vaporizable material, the user's (of the vaporizer device 100) preferences or desired experience, and/or the like.

Data read from the tag 164 from the wireless communication circuitry 142 of the vaporizer body 110 may be transferred to one or more of the remote processors (e.g., the user device 305 and/or the remote server 307) to which it is connected, which allows for the app running on the one or more processors to access and utilize the read data for a variety of purposes. For example, the read data relating to the cartridge 150 may be used for providing recommended temperatures, dose control, usage tracking, and/or assembly information.

The cartridge 150 may also communicate directly, through the tag 164, with other devices. This enables data relating to the cartridge 150 to be written to/read from the tag 164, without interfacing with the vaporizer body 110. The tag 164 thus allows for identifying information (e.g., pod ID, batch ID, etc.) related to the cartridge 150 to be associated with the cartridge 150 by one or more remote processors. For example, when the cartridge 150 is filled with a certain type of vaporizable material, this information may be transmitted to the tag 164 by filling equipment. Then, the vaporizer body 110 is able to obtain this information from the tag 164 (e.g., via the wireless communication circuitry 142 at the vaporizer body 110) to identify the vaporizable material currently being used and accordingly adjust the controller 128 based on, for example, user-defined criteria or pre-set parameters associated with the particular type of vaporizable material (set by a manufacturer or as determined based upon user experiences/feedback aggregated from other users). For example, a user may establish (via the app) a set of criteria relating to desired effects for or usage of one or more types of vaporizable materials. When a certain vaporizable material is identified, based on communication via the tag 164, the controller 128 may accordingly adopt the established set of criteria, which may include, for example, temperature and dose, for that particular vaporizable material.

Consistent with implementations of the current subject matter, the vaporizable material used with the vaporizer device may be provided within the cartridge. The vaporizer device may be a cartridge-using vaporizer device, a cartridge-less vaporizer device, or a multi-use vaporizer device capable of use with or without a cartridge. For example, a multi-use vaporizer device may include a heating chamber (e.g., an oven) configured to receive the vaporizable material directly in the heating chamber and also configured to receive the cartridge having a reservoir or the like for holding the vaporizable material. In various implementations, the vaporizer device may be configured for use with liquid vaporizable material (e.g., a carrier solution in which an active and/or inactive ingredient(s) are suspended or held in solution or a liquid form of the vaporizable material itself) or solid vaporizable material. Solid vaporizable material may include a plant material that emits some part of the plant material as the vaporizable material (e.g., such that some part of the plant material remains as waste after the vaporizable material is emitted for inhalation by a user) or optionally may be a solid form of the vaporizable material itself such that all of the solid material may eventually be vaporized for inhalation. Liquid vaporizable material may likewise be capable of being completely vaporized or may include some part of the liquid material that remains after all of the material suitable for inhalation has been consumed.

As described above, the vaporizer device 100 and/or the user device 305 that is part of a vaporizer system as defined above may include a user interface (e.g., including an app or application software) that may be executed on the user device 305 in communication, which may be configured to determine, display, enforce, and/or meter dosing.

Aspects of the current subject matter relating to vaporizer device configurations including controlling operation of a vaporizer device with respect to dose consumption are not limited to use with the particular configurations and/or components of the vaporizer device 100, the vaporizer body 110, and the cartridge 150. Rather, aspects of the current subject matter may be employed with various other vaporizer devices, vaporizer bodies, and cartridges and/or with various modifications of the vaporizer device 100, the vaporizer body 110, and the cartridge 150. For example, consistent with implementations of the current subject matter, aspects of the current subject matter may be employed without the tag 164 of the cartridge 150 and/or the wireless communication circuitry of the vaporizer body 110. Consistent with implementations of the current subject matter, the vaporizer device 100 need not communicate with the user device 305, and accordingly, components that facilitate such communication are not required. Moreover, various sensors and circuitry are not required for the dose consumption control operations provided herein. For example, the ambient pressure sensor 138, the accelerometer 139, and/or the cartridge detection circuitry 148 are not required in some implementations. Various other combinations of configurations and/or components of the vaporizer device 100, the vaporizer body 110, and the cartridge 150 may be employed consistent with implementations of the current subject matter.

According to aspects of the current subject matter, a dose is defined as a fixed amount of aerosol generated by the vaporizer device 100 for consumption by the user as a number of puffs taken by the user until the fixed amount of aerosol is consumed or inhaled. Consistent with implementations of the current subject matter, the fixed amount of aerosol to be delivered to the user may be based on an amount of energy used to produce vapor from the vaporizable material. Consistent with implementations of the current subject matter, an energy value may be based on total particulate matter, which refers to the amount of vaporizable material removed from the cartridge 150 (e.g., from a wicking element of the heater 166) by vaporization or aerosolization and suspended in the vapor for consumption by the user. For example, there is a correlation between the amount of energy supplied and an amount of vaporizable material removed from the cartridge 150.

In some implementations, the amount of energy is a fixed amount. In some implementations, the amount of energy may be one factor in combination with additional factors to determine the fixed amount of aerosol to be delivered to the user. The additional factors may include, for example, a preset or predetermined temperature for the dose, characteristics or properties of the vaporizable material (such as viscosity, age or date of production, chemical composition, concentrations, etc.), and/or usage data (such as date of production of the cartridge 150, frequency of use of the cartridge 150, date and time of last use of the cartridge 150, number of doses completed, etc.). Various fixed amounts (e.g., dose sizes) may be defined such that the fixed amount of aerosol corresponds to, for example, an amount of energy to be sent to the heater 166. In other implementations, the dose sizes may be defined by and/or based on energy applied before and/or at the start of each user puff and/or temperature at the start and/or end of each user puff.

Aspects of the current subject matter provide for allowing a user to monitor and/or control consumption of the vaporizable material. Consistent with implementations of the current subject matter, the vaporizer device 100 may provide feedback to the user with respect to dose consumption of the vaporizable material being vaporized and inhaled by the user.

For example, in accordance with implementations of the current subject matter, the vaporizer device 100 may provide feedback indicative of consumption of one or more doses of the vaporizable material being vaporized and inhaled by the user. The feedback may be in the form of, for example, a dose pulse such as a haptic pulse and/or haptic pattern generated by the haptics system 144, to signify consumption of each dose, where a dose may be defined as a fixed unit of vapor. Additionally or alternatively, the feedback may be provided via audio and/or visual feedback. For example, the feedback may be via the LEDs 136, such as a predetermined animated pattern displayed via the LEDs 136 to signify consumption of each dose or a number of doses. Additionally or alternatively, the feedback may be provided as a type of audio and/or visual representation on the user interface of the user device 305. The type of feedback and characteristics of the feedback may be user defined and/or configurable, and/or may be based on the vaporizer device 100 and/or the cartridge 150. For example, the type of feedback and its corresponding characteristics may be selected and/or adjusted by the user. In some implementations, the feedback may be a unique form of feedback for dose consumption indication; for example, a unique haptic signal (e.g., haptic pulse and/or haptic pattern), audio signal, visual signal, or combination thereof that is meant to signify dose consumption. Moreover, a unique form of feedback may be provided at various points during consumption as described herein; for example, a first unique form of feedback when dose consumption is initiated, a second unique form of feedback during dose consumption, and a third unique form of feedback when dose consumption is completed. In some implementations, the feedback during dose consumption may correspond to various factors, such as the number of doses consumed and/or the number of doses remaining, as further described herein.

Consistent with some implementations of the current subject matter, if a particular dose is only partially consumed (e.g., the user draws up to, for example, 99% of the dose), the partial consumption of the dose may count as a full dose if the partial consumption meets or exceeds a predetermined amount (e.g., greater than 50%, 60%, 70%, 80%, 90%, 95%, or other defined value). The predetermined amount for partial dose consumption may be user defined and configurable (e.g., through the user device 305) and may be associated with the cartridge 150, the vaporizer device 100, or both. If the partial consumption meets or exceeds the predetermined amount, the feedback is outputted by the vaporizer device 100. Thus, if the user consumes the amount of the dose defined by the predetermined amount, the vaporizer device 100 responds by outputting feedback as if a full dose were consumed. This prevents the situation of the user beginning consumption of a new dose, followed by an earlier partial dose that was at or exceeding the predetermined amount, and the vaporizer device 100 responding with the feedback.

Implementations of the current subject matter provide for various dose control modes or settings, the selection of which provides for a particular type of dose consumption feedback. For example, a continuous dose control mode and a series dose control mode may be provided by the vaporizer device 100. The type of mode may be inputted by the user via the user device 305 or via the vaporizer device 100. The type of mode may be preconfigured and associated with the vaporizer device 100 and/or the cartridge 150. For example, a particular type of cartridge 150 may have a particular dose control mode associated with the cartridge 150, and/or a particular vaporizer device 100 may have a particular dose control mode associated with the vaporizer device 100. The dose control mode may be user-configurable and/or user-adjustable. In some implementations, the dose control mode may not be altered. In some implementations, the dose control mode may be altered by an authorized user and/or via an authenticated signal (e.g., a signal from an authenticated user device 305 transmitted to the cartridge 150 and/or the vaporizer body 110). During the continuous dose control mode and the series dose control mode, active consumption of a dose (e.g., when the user is inhaling on the vaporizer device 100) may be represented by, for example, the animated display of the LEDs 136.

Consistent with implementations of the current subject matter, the continuous dose control mode allows for the user to consume an undefined number of doses with each dose consumed being signified by one or more types of feedback, such as the dose pulse, the animated display of the LEDs 136, and/or the audio and/or visual representation on the user interface of the user device 305. Consistent with implementations of the current subject matter, feedback may be outputted by the vaporizer device 100 when the dose control mode is activated and/or deactivated (e.g., dose pulse by the haptics system 144 and/or animated display of the LEDs 136). The undefined number of doses may refer to an unlimited number of doses and/or a number of doses not limited to a particular value. For example, the undefined number of doses may allow the user to consume any number of doses without affecting operation of the vaporizer device 100.

Consistent with implementations of the current subject matter, if a particular dose is only partially consumed (e.g., the user draws up to, for example, 99% of the dose), when the user begins to inhale on the vaporizer device 100 at a later time that exceeds a timer or reset value, this action may be counted as a new dose in the continuous dose control mode. In accordance with implementations of the current subject matter, data indicative of partial dose consumptions may be collected and stored (e.g., on the vaporizer device 100 and/or on the tag 164 of the cartridge 150) for further analysis and data collection. In some implementations, the timer or reset value may be defined to indicate a time at which the continuous dose control mode becomes inactive. For example, if the timer or reset value is reached following a user's puff, when the user puffs again, the continuous dose control mode may be re-initiated or reactivated. Thus, if the vaporizer device 100 has not been used for a period of time greater than the timer or reset value, the user will not receive the one or more types of feedback before a new dose is consumed.

Consistent with implementations of the current subject matter, the series dose control mode provides for the user to set or select a limited number of doses to be consumed, with each dose consumed being signified or represented by one or more types of feedback, such as the dose pulse, the animated display of the LEDs 136, and/or the audio and/or visual representation on the user interface of the user device 305. Consistent with implementations of the current subject matter, feedback may be outputted by the vaporizer device 100 when the series dose control mode is activated and/or deactivated (e.g., dose pulse and/or animated display of the LEDs 136), serving as an indicator to alert the user. An end of series feedback may be outputted by the vaporizer device 100 when the series is completed, for example, when the set limited number of doses to be consumed is reached. This may serve as a representation to the user that the series is completed (e.g., that the set limited number of doses to be consumed is reached). Moreover, consistent with implementations of the current subject matter, the end of series feedback may also signify the number of consumed doses in the series (e.g., three long pulses for three consumed doses, three long flashes of the LEDs 136 for three consumed doses, etc.).

The series dose control mode may incorporate a timeout period signifying an amount of time that needs to elapse between series of doses, such as consecutive series of doses. For example, according to some aspects, once the series dose of the limited number of doses is completed, the vaporizer device 100 may be locked for a preset amount of time (referred to as the timeout period or as a lockout period or a predefined lockout period). The amount of time for the timeout period may be user-defined and/or user-configurable (e.g., set by the user using the app through the user device 305). For example, the user may set the timeout period when selecting the series dose control mode. The timeout period may be associated with the cartridge 150 and/or the vaporizer device 100. The timeout period may be stored on the tag 164 of the cartridge 150 and read by the wireless communication circuitry 142 of the vaporizer body 110, or provided by a user device (e.g., the user device 305) or a remote server (e.g., the remote server 307). A default timeout period may be defined, and may be associated with the cartridge 150 and/or the vaporizer device 100, and may in some instances be adjusted by the user. In some implementations of the current subject matter, the default timeout period is not adjustable. Consistent with implementations of the current subject matter, the timeout period may be signified to the user by timeout feedback outputted by the vaporizer device 100 in the form of, for example, one or more haptic pulses by the haptics system 144 and/or an animated display of the LEDs 136. Various stages of the timeout period (e.g., a start of the timeout period, a duration of the timeout period, and an end of the timeout period) may have corresponding timeout feedback outputted.

During the timeout period, in some implementations, user puffing does not produce any vapor. For example, during the timeout period the vaporizer device 100 does not allow activation of the heating element. During the timeout period, consistent with implementations of the current subject matter, if the user puffs or draws on the mouthpiece, vapor is not produced. Once the timeout period ends, parameters of the series dose control mode (e.g., the limited number of doses to be consumed and/or the timeout period) may be set as the same as the previous series unless otherwise updated by the user. There may be an option to override and/or end the timeout period. If such an option is selected, the vaporizer device 100 is provided with data or a signal indicative of the user selection to override and/or end the timeout period, and the vaporizer device 100 may then accordingly respond to the user puffing or drawing on the mouthpiece and/or to selection of a new series. In some implementations, the timeout period may not be overridden by the user. In some implementations, the user or a manufacturer may establish settings regarding use of the timeout period. For example, the user or the manufacturer may establish a certain number of timeout periods that may be overridden in a given time period.

The series dose control mode consistent with implementations of the current subject matter may also incorporate a timer value that indicates a maximum amount of time between doses in the series before the series resets. For example, in the series dose control mode when the user initiates a series by inhaling on the vaporizer device 100, the user may have an amount of time indicated by the timer value to consume the next dose in the series.

Consistent with implementations of the current subject matter, the timer value may be defined to cover the time for a complete series. For example, for a four dose series size, the timer value may indicate the amount of time in which the user has to complete the four doses before the series is reset. The series may start at the time of the first inhale by the user on the vaporizer device 100, and the series may end at the consumption of the last dose. If the user has not completed all of the doses in the series in an amount of time indicated by the timer value, the series may be reset.

In some implementations, two timer values may be incorporated. A first timer value may be used to define a maximum amount of time between doses in the series before the series resets, and a second timer value may be used to define a maximum amount of time in which the series needs to be completed. In some implementations, the timer value is defined as the amount of time between doses in the series before the series ends or is reset.

If the amount of time between doses consumed exceeds the timer value, then, according to implementations of the current subject matter, the series may be reset and a new series started. The timer value may be user or system defined (e.g., by data provided by the remote server 307 and/or the user device 305), similar to the timeout period. For example, the user may set the timer value when selecting the series dose control mode (and associated settings such as the number of doses for a particular series). The timer value may be defined by a filler or provider of the vaporizable material and/or the cartridge 150, or by a third party such as a caregiver or other third party. The timer value may be associated with the cartridge 150 and/or the vaporizer device 100. The timer value may be stored on the tag 164 of the cartridge 150 and read by the wireless communication circuitry 142 of the vaporizer body 110. A default timer value may be defined, and may be associated with the cartridge 150 and/or the vaporizer device 100, and may be adjusted by the user. In some implementations of the current subject matter, the default timer value is not adjustable.

Consistent with implementations of the current subject matter, dose control settings, such as selecting a particular dose control mode, may be enabled, changed, and/or disabled via an app running on the user device 305 or through other pre-defined action (e.g., a particular movement or series of movements of the vaporizer device 100 and/or the cartridge 150).

In accordance with implementations of the current subject matter, dose control mode and dose control settings may be associated with and persistent to the cartridge 150. For example, if the dose control mode is activated and dose control settings established for a particular cartridge 150, removal of the cartridge 150 results in the dose control mode and the dose control settings being applied to the cartridge 150 upon reinsertion of the cartridge 150. If another cartridge 150 has the dose control mode and dose control settings associated with it, the associated dose control mode and dose control settings for that cartridge 150 may be applied upon insertion. The association to the cartridge 150 may be achieved by storing the dose control mode and dose control settings to the cartridge 150 with the tag 164. For example, the dose control mode and dose control settings, including progress or current point of the dose control mode, are stored on the tag 164 through data transmission with the wireless communication circuitry 142.

Alternatively, the vaporizer device 100 and the cartridge 150 may reset to baseline parameters, which may be user or system defined and/or customizable and which may, in some instances, be overridden. To deactivate the dose control mode and/or the dose control settings, the user may be required to, for example, access the app on the user device 305. Other user-controlled actions may serve to deactivate the dose control mode and/or the dose control settings. For example, a particular movement or series of movements of the vaporizer device 100, the cartridge 150, and/or the cartridge 150 with respect to the vaporizer device may be used to activate and/or deactivate dose control settings.

In some implementations, dose control activation, dose control mode, and dose control settings may be lost upon removal of the cartridge 150. For example, removal of the cartridge 150 followed by a complete goodbye animation (e.g., an animation of the LEDs 136 signifying the complete removal of the cartridge 150 as opposed to a rapid remove and insert process) or a period of time that equals to or exceeds a value indicating a goodbye or end of use phase, may indicate deactivation. Deactivation of dose control may also or alternatively be established via the app on the user device 305. Consistent with implementations of the current subject matter, dose control is a persistent mode until explicitly deactivated.

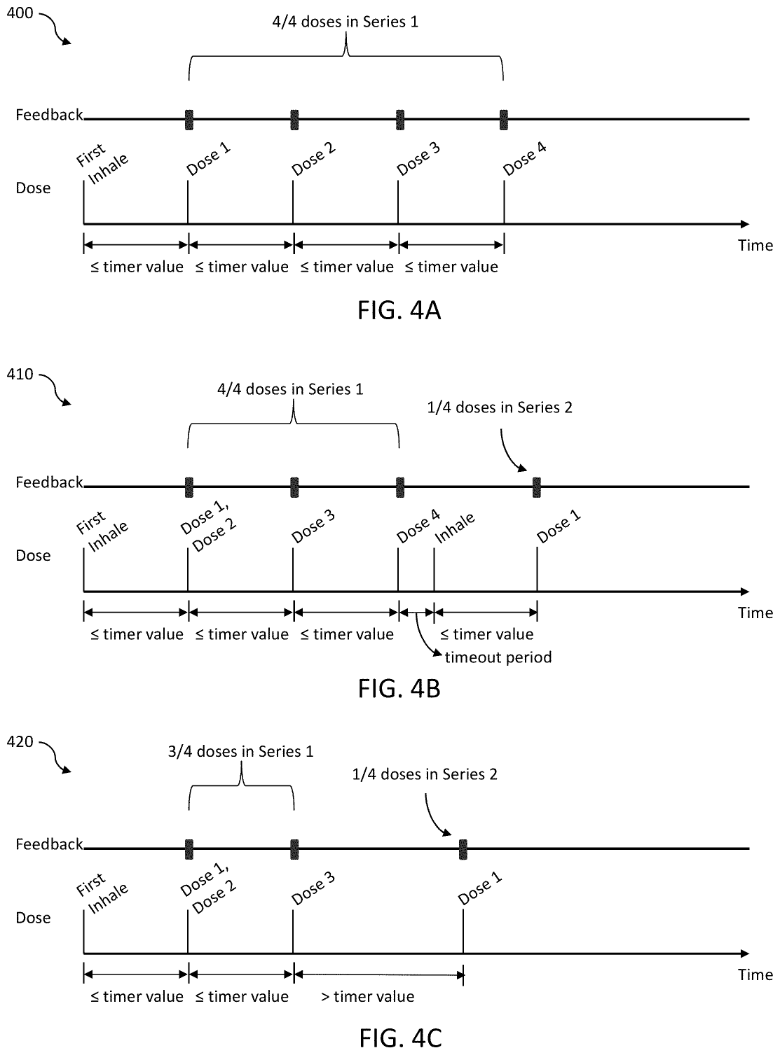

With reference to FIG. 4A-FIG. 4C, timing and dose feedback aspects of the vaporizer device 100 for the series dose control mode, consistent with implementations of the current subject matter, are illustrated.

Diagram 400 in FIG. 4A illustrates aspects of an example of a four dose series size, although other size series may be used as well. As shown, the user has a first inhale followed by completion of dose one within a time period less than a timer value, the timer value signifying the maximum amount of time between doses before the series is reset. Similarly, each of dose two, dose three, and dose four are completed within periods of time less than the timer value. The four doses are part of a single dose series (series 1). Also illustrated in FIG. 4A are the outputted feedbacks at the time of completion of dose one, dose two, dose three, and dose four.

Diagram 410 in FIG. 4B illustrates aspects of an example of a four dose series size of the series dose mode of the vaporizer device 100. As shown, the user completes dose one, dose two, dose three, and dose four within periods of time less than the timer value. Accordingly, the first four doses are part of a single dose series (series 1). Following the fourth dose of the four dose series, a timeout period is applied. At the end of the timeout period or at some point thereafter, the user inhales on the vaporizer device 100 and completes another first dose within a time period less than the timer value. This first dose is the first of four possible doses in series 2. Also illustrated in FIG. 4B are the outputted feedbacks at the time of completion of dose one, dose two, dose three, and dose four in series 1 and dose one in series 2.

Diagram 420 in FIG. 4C illustrates aspects of an example of a four dose series size of the series dose mode of the vaporizer device 100. As shown, the user completes dose one, dose two, and dose three within periods of time less than the timer value. Accordingly, the first three doses are part of a single dose series (series 1). Following the third dose, the user inhales and completes the next dose within a time period greater than the timer value. Thus, the next dose is the first dose of the following series, series 2. This dose is the first of four possible doses in series 2. Also illustrated in FIG. 4C are the outputted feedbacks at the time of completion of dose one, dose two, and dose three in series 1 and dose one in series 2.

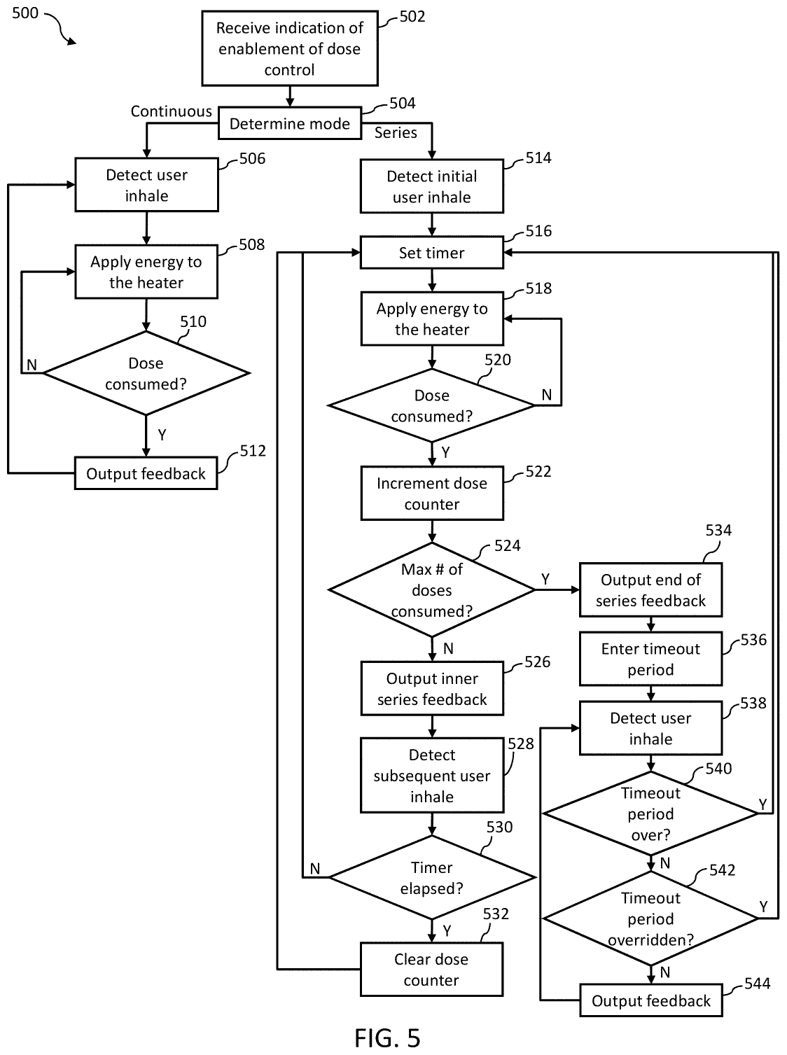

With reference to FIG. 5, a chart 500 illustrates features of a method, which may optionally include some or all of the following.

At 502, the vaporizer device 100 receives an indication of enablement of dose control. The vaporizer device 100 may, consistent with implementations of the current subject matter, receive the indication of enablement of dose control from the user device 305. For example, the user may utilize an app running on the user device 305 to select or otherwise indicate that the user wishes to enable the dose control mode and the dose control settings consistent with implementations of the current subject matter. The user may additionally or alternatively manipulate the vaporizer device 100 (e.g., remove and reinsert the cartridge 150, shake the vaporizer device 100, etc.) to enable the dose control mode and the dose control settings consistent with implementations of the current subject matter. The user may wish to be kept informed of the number of doses being consumed and/or want to set a limited number of doses to be consumed. The user may also wish to set dose control settings related to the timeout period between the series of doses and/or the timer value that signifies the maximum amount of time between doses before the dose series is reset. The dose control settings may also or alternatively include the desired number of doses and/or a size of each of the doses. In some implementations, the size of the doses may be a constant value or may vary. For example, the dose size may increase for each subsequent dose for the desired number of doses, or may decrease for each subsequent dose. In some implementations, the dose sizes may be any dose size from a selection of dose sizes and may vary throughout the desired number of doses.

At 504, the dose control mode is determined. For example, the dose control mode may be provided to the vaporizer device 100 from the user device 305 upon selection of the dose control mode by the user via the app. As described herein, implementations of the current subject matter provide for the continuous dose control mode and the series dose control mode. The continuous dose control mode allows for the user to consume an undefined and/or unlimited number of doses (e.g., not limited to a particular value), with each dose consumed being signified or represented by one or more types of feedback, such as the dose pulse from the haptics system 144, the animated display of the LEDs 136, and/or the audio and/or visual representation on the user interface of the user device 305. The series dose mode allows for the user to set a limited number of doses to be consumed, with each dose consumed being signified by one or more types of feedback, such as the dose pulse, the animated display of the LEDs 136, and/or the audio and/or visual representation on the user interface of the user device 305. Consistent with implementations of the current subject matter, feedback (e.g., activation feedback in the form of the dose pulse and/or animated display of the LEDs 136) may be outputted by the vaporizer device 100, when the continuous dose control mode or the series dose control mode is activated.

Consistent with implementations of the current subject matter, the enablement of dose control (502) and/or the determination of the dose control mode (504) may be achieved by reading data associated with the cartridge 150. For example, the dose control mode for a particular cartridge 150 may be stored on the tag 164 of the cartridge 150 and read by the vaporizer device 100 after the cartridge 150 is inserted into the vaporizer body 110.

At 506, if the determined dose control mode (at 504) is the continuous dose control mode, the vaporizer device 100 may detect a user inhale on the vaporizer device 100. The detection of the user inhale may be based on various factors, for example, pressure changes and/or sensor readings. Consistent with implementations of the current subject matter, the detection of the user inhalation is representative of a start to a dose being consumed by the user.

At 508, in response to the detection of the user inhale (at 506), the vaporizer device 100 applies energy to the heater. For example, the heater control circuitry 130 of the vaporizer body 110 may control the heater 166 of the cartridge 150 to generate heat to provide vaporization of the vaporizable material, where the energy applied is correlated with the dose size.

At 510, a determination is made by the vaporizer device as to whether the user has consumed the dose. For example, consistent with implementations of the current subject matter, the dose may be defined as a fixed unit of vapor and may be based on the amount of energy used to produce vapor from the vaporizable material. For example, the amount of energy is related to an amount of vapor produced. In some implementations, dose may be based on an amount of energy applied to the heater 166 (e.g., how much heat is applied). In some implementations, dose may be based on an amount of power applied to the heater 166, a voltage applied to the heater 166, a current applied to the heater 166, a resistance applied to the heater 166, or combinations thereof, although dose may be determined in other ways. In some implementations, dose is based on the energy applied to the heater 166 over a time period, the power applied to the heater 166 over a time period, the voltage applied to the heater 166 over a time period, the current applied to the heater 166 over a time period, the resistance applied to the heater 166 over a time period, air path pressure, or combinations thereof. In some implementations, a partial consumption of a dose may correlate to consumption of the dose if the partial consumption meets or exceeds a predetermined amount. The predetermined amount may be defined as a percentage, and may be a percentage of the energy applied to amount to a full dose. For example, if the predetermined amount is defined as 95%, if 95% of the energy is applied, the dose may be counted as a full dose.

If, as determined at 510, the dose has not been consumed, the vaporizer device 100 continues to apply energy to the heater 166. If instead, as determined at 510, the dose has been consumed by the user, then at 512 the vaporizer device 100 outputs the feedback to signify to the user that the dose has been consumed. For example, the dose pulse generated, for example, by the haptics system 144, the animated display of the LEDs 136, and/or the audio and/or visual representation on the user interface of the user device 305 may be outputted.

In the continuous dose control mode, the process of detecting the user inhale (at 506), applying energy to the heater (at 508), determining consumption of the dose (at 510), and outputting the feedback (at 512) may continue during use of the vaporizer device 100 until the user deactivates the continuous dose control mode. For example, the user may deactivate the continuous dose control mode though the app, by removing the cartridge 150, and/or by activating the series dose control mode.

With continued reference to FIG. 5, at 514, if the determined dose control mode (at 504) is the series dose control mode, the vaporizer device 100 may detect a user inhale on the vaporizer device 100. The detection of the user inhale may be based on various factors, for example, pressure changes and/or sensor readings. Consistent with implementations of the current subject matter, the detection of the user inhalation may be representative of a start to a dose being consumed by the user.

At 516, upon detection of the user inhale (at 514), the vaporizer device 100 sets a timer. Consistent with implementations of the current subject matter, the series dose control mode incorporates the timer value as a maximum amount of time between doses in the series before the series resets. The time value feature consistent with implementations of the current subject matter may be desirable to users to control or allow an initiation of a new series based on the amount of time between dose consumptions. For example, if a significant amount of time passes between dose consumptions, the user may not want the dose consumptions that span across the significant amount of time to count as consecutive doses in a series. Thus, the time value feature consistent with implementations of the current subject matter allows for the series to be reset so that a new series of doses may be initiated. The timer value may be user or system defined. For example, the user may set the timer value when selecting the series dose control mode. The timer value may be associated with the cartridge 150 and/or the vaporizer device 100. The timer value may be stored on the tag 164 of the cartridge 150 and read by the wireless communication circuitry 142 of the vaporizer body 110. A default timer value may be defined, and may be associated with the cartridge 150 and/or the vaporizer device 100, and may be adjusted by the user.

At 518, the vaporizer device 100 applies energy to the heater 166. For example, the heater control circuitry 130 of the vaporizer body 110 may control the heater 166 of the cartridge 150 to generate heat to provide vaporization of the vaporizable material.

At 520, a determination is made by the vaporizer device 100 as to whether the user has consumed the dose. For example, consistent with implementations of the current subject matter, the dose may be defined as a fixed unit of vapor and may be based on the amount of energy used to produce vapor from the vaporizable material. For example, the amount of energy is related to an amount of vapor produced. In some implementations, dose may be based on an amount of energy applied to the heater 166 (e.g., how much heat is applied). In some implementations, dose may be based on an amount of power applied to the heater 166, a voltage applied to the heater 166, a current applied to the heater 166, a resistance applied to the heater 166, or combinations thereof. In some implementations, dose is based on the energy applied to the heater 166 over a time period, the power applied to the heater 166 over a time period, the voltage applied to the heater 166 over a time period, the current applied to the heater 166 over a time period, the resistance applied to the heater 166 over a time period, or combinations thereof. In some implementations, a partial consumption of a dose may correlate to consumption of the dose if the partial consumption meets or exceeds a predetermined amount. The predetermined amount may be defined as a percentage, and may be a percentage of the energy applied to amount to a full dose. For example, if the predetermined amount is defined as 95%, if 95% of the energy is applied, the dose may be counted as a full dose.

If, as determined at 520, the dose has not been consumed, the vaporizer device 100 continues to apply energy to the heater 166. If instead, as determined at 520, the dose has been consumed by the user, then at 522 a dose counter is incremented. As the series dose control mode defines a number of doses to be consumed in the series, the vaporizer device 100 needs to track the number of doses consumed to determine where the user is in the series. For example, if the user is at the end of the series, the end of series feedback may be outputted and a timeout period initiated. If the user is not at the end of the series, the outputted feedback may be specific to the number of doses consumed in the series and/or the number of doses remaining in the series. In some instances of the current subject matter, the vaporizer device 100 may track the number of doses consumed by monitoring and/or counting energy consumption (e.g., an energy value in milliJoules or other unit). The monitoring and/or counting of energy consumption may be an alternative to or an addition to tracking and/or counting a number of doses.

At 524, the vaporizer device 100 determines, based on the incremented dose counter and the limited number of doses defined or established for the series dose control mode, if the maximum number of doses for the series dose control mode have been consumed. For example, once a dose is consumed by the user, the dose counter is incremented to represent the number of doses in the series consumed by the user. This value is compared to the limited number of doses that were defined for the series. In this manner, the vaporizer device 100 tracks the number of doses consumed in the series and the number of doses remaining in the series. According to aspects of the current subject matter, the vaporizer device 100 may track the doses by monitoring and/or counting energy consumption rather than or in addition to tracking and/or counting a number of doses.

At 526, an inner series feedback is outputted by the vaporizer device 100. Consistent with implementations of the current subject matter, the inner series feedback may be the feedback to represent the consumption of a dose in the series. The outputting of the inner series feedback is based on a determination (at 524) that the maximum number of doses in the series have not been consumed. The vaporizer device 100 outputs the feedback to signify to the user that the dose has been consumed. For example, the dose pulse generated, for example, by the haptics system 144, the animated display of the LEDs 136, and/or the audio and/or visual representation on the user interface of the user device 305 may be outputted. The inner series feedback may signify a proportion of doses consumed and/or available to be consumed within the current series (e.g., one short pulse for one consumed dose followed by three short pulses to signify three remaining doses, etc.).

At 528, the vaporizer device 100 may detect a subsequent user inhale on the vaporizer device 100. The detection of the subsequent user inhale may be based on various factors, for example, pressure changes and/or sensor readings. Consistent with implementations of the current subject matter, the detection of the user inhalation may be representative of a start to a dose being consumed by the user.

At 530, following the detection of the subsequent user inhale at 528, a determination is made as to whether the timer value, set at 516, has elapsed. This determination may be made to determine if there is time remaining in the series (as defined by the timer value) so that the next dose being consumed by the user is part of the current series of doses. If there is no time remaining in the series (as defined by the timer value), then the next dose being consumed by the user is part of a new series of doses. Thus, the timer value may, consistent with implementations of the current subject matter, be used to determine if the series of doses should be cleared, for example, if a new series of doses should be started. As an example, if a significant amount of time (e.g., equal to or greater than that of the timer value) has passed between dose consumptions, the series of doses is cleared so that the user may start another series of doses. If, however, there is still time remaining between doses, the series of doses continues and the next dose being consumed is part of the current series.

If the determination at 530 is that the timer value has not elapsed, the process may return to 516 to reset the timer. In this instance, the dose counter is not cleared, and the next dose being consumed is part of the current series of doses. If, however, the determination at 530 is that the timer value has elapsed, this signifies that a new series of doses needs to be started.

At 532, if, as determined at 530, that the timer value has elapsed, the dose counter is cleared. The clearing of the dose counter, consistent with implementations of the current subject matter, provides for a new series of doses as part of the series dose control mode. As described herein, the timer value elapsing may signify that the series of doses should be reset due to the amount of time between subsequent dose consumptions by the user. Following the clearing of the dose counter (at 532), the process returns to 516 to reset the time (e.g., the timer starts again to indicate the amount of time between doses). As a new dose is being consumed, in a new series, the resetting or clearing of the timer value is done to coincide or align with the start of the new dose and may be used to determine if subsequent doses are part of the series. In some implementations, once the dose counter is cleared (at 532), a period of time (e.g., the timeout period) may be applied during which a new dose and new series cannot be initiated.

Still referring to FIG. 5, at 534, the end of series feedback is outputted based on a determination (at 524) that the maximum number of doses in the series has been consumed. The outputting of the end of series feedback signifies to the user that the last dose in the series has been consumed and that the series is over. The end of series feedback may, consistent with implementations of the current subject matter, include the dose pulse generated, for example, by the haptics system 144, the animated display of the LEDs 136, and/or the audio and/or visual representation on the user interface of the user device 305 may be outputted. Consistent with implementations of the current subject matter, the end of series feedback may differ from the inner series feedback and may signify, for example, the number of consumed doses in the series and/or other detail related to the series, for example, length of time (e.g., three long pulses for three consumed doses, three long flashes of the LEDs 136 for three consumed doses, etc.).

At 536, the timeout period is entered. Consistent with implementations of the current subject matter, the series dose control mode may incorporate the timeout period as an amount of time during which the vaporizer device 100 is locked between series. The amount of time for the timeout period may be user or system defined. For example, the user may set the timeout period when selecting the series dose control mode. The timeout period may be associated with the cartridge 150 and/or the vaporizer device 100. The timeout period may be stored on the tag 164 of the cartridge 150 and read by the wireless communication circuitry 142 of the vaporizer body 110. A default timeout period may be defined, and may be associated with the cartridge 150 and/or the vaporizer device 100, and may be adjusted by the user. In some implementations of the current subject matter, the default timeout period is not adjustable. Consistent with implementations of the current subject matter, the timeout period may be signified to the user by timeout feedback outputted by the vaporizer device in the form of, for example, one or more haptic pulses and/or an animated display of the LEDs 136. Various stages of the timeout period (e.g., a start of the timeout period, a duration of the timeout period, and an end of the timeout period) may have corresponding timeout feedback outputted.

At 538, the vaporizer device 100 detects a user inhale based on various factors, for example, pressure changes and/or sensor readings. As the vaporizer device 100 is in the timeout period, the vaporizer device 100 may not respond to the user inhale and/or may provide feedback, such as a haptics pulse, LED animation, and/or audio and/or visual output, to alert the user that a dose cannot be consumed.

At 540, the vaporizer device 100 determines if the timeout period has elapsed. If the timeout period is over, the process may continue to 516 to reset the timer and start a new series of doses. Based on the vaporizer device 100 having been in the timeout period, the vaporizer device 100 may clear the dose counter.

At 542, a determination is made as to whether the timeout period is overridden. This determination may be in response to the user inhaling (at 538) on the vaporizer device and the determination (at 540) that the timeout period is not over. The timeout period may, consistent with implementations of the current subject matter, be overridden by the user via the app or by a predefined action with respect to the vaporizer device 100 (e.g., a rapid removal and insert of the cartridge or other action detectable by the vaporizer device 100). If the timeout period is overridden, the process may continue to 516 to reset the timer and start a new series of doses. Based on the vaporizer device 100 having been in the timeout period, the vaporizer device 100 may clear the dose counter.

At 544, the vaporizer device 100 may output feedback to signify to the user that the vaporizer device 100 remains in the timeout period. For example, the outputted feedback may be in the form of a haptics pulse, an animated display of the LEDs 136, and/or an indication on the user device 305. Following the outputted feedback, the process may continue such that the vaporizer device 100 detects the user inhale (at 538) and proceeds with evaluation of the timeout period. Consistent with implementations of the current subject matter, once the timeout period has elapsed, the vaporizer device 100 may be in a standby mode (e.g., at 514) to implement the series dose control mode when consumption of a dose is initiated.

As described herein, the outputted feedback from the vaporizer device may include pulses generated by the haptics system 144 and/or an animated display of the LEDs 136.

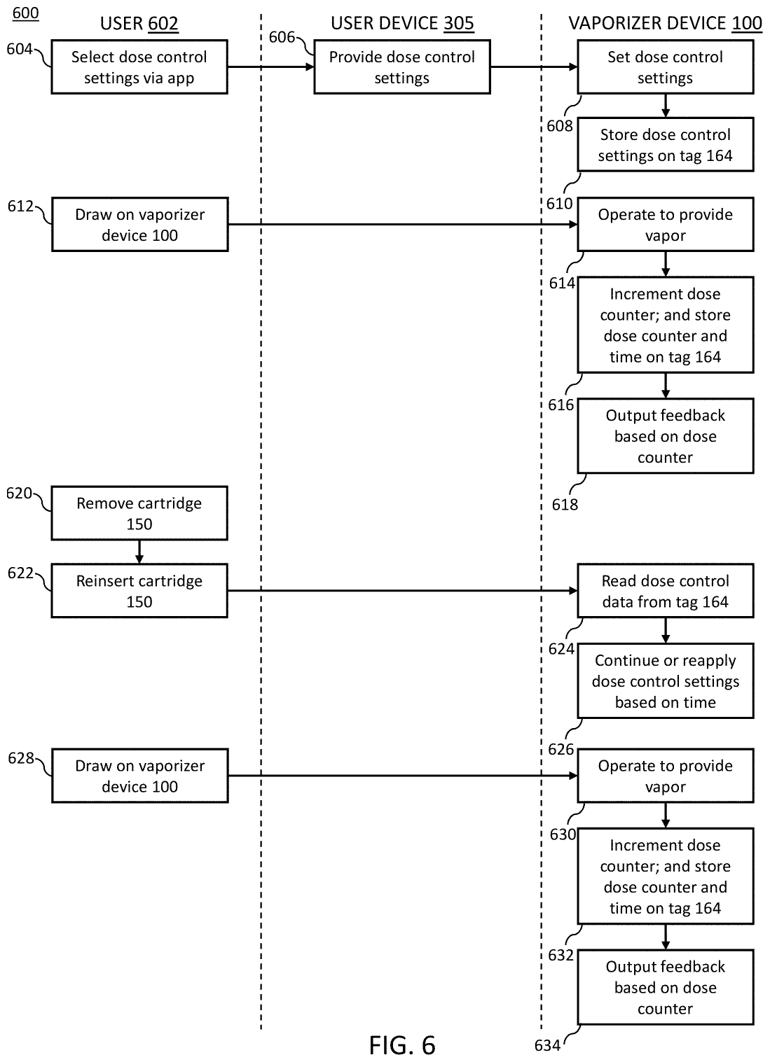

FIG. 6 shows a swim lane diagram 600 illustrating operations of a user 602, the user device 305, and the vaporizer device 100 consistent with implementations of the current subject matter.

At 604, the user 602 selects the dose control mode and/or the dose control settings via the app running on the user device 305. For example, the user may utilize the app running on the user device 305 to select or otherwise indicate that the user wishes to enable the dose control mode and the dose control settings consistent with implementations of the current subject matter. The user 602 may select the continuous dose control mode or the series dose control mode. The continuous dose control mode allows for the user 602 to consume an undefined and/or unlimited (e.g., not limited to a particular value) number of doses, with each dose consumed being signified by one or more types of feedback. The series dose mode allows for the user 602 to set a limited number of doses to be consumed, with each dose consumed being signified by one or more types of feedback. Consistent with implementations of the current subject matter, feedback (e.g., activation feedback in the form of the dose pulse and/or animated display of the LEDs 136) may be outputted by the vaporizer device 100 when the continuous dose control mode or the series dose control mode is activated. The settings may be received by the user device 305 and/or the app (e.g., as an indication of the selection made via the app).

At 606, the dose control settings, including the dose control mode, are provided from the user device 305 to the vaporizer device 100. As the user 602 enters the dose control mode and/or the dose control settings via the app running on the user device 305, the user device 305 then needs to transmit this information to the vaporizer device 100 to provide for the vaporizer device 100 to implement the correct dose control mode. At 608, upon receipt of the dose control settings, the vaporizer device 100 sets the dose control settings.

At 610, the vaporizer device 100 may store the dose control settings, including the dose control mode, on the tag 164 of the cartridge 150. This allows for the dose control mode and/or the dose control settings to be associated with the cartridge 150. For example, consistent with implementations of the current subject matter, the vaporizer device 100 may read the dose control mode and/or the dose control settings from the tag 164 for subsequent dose control operations.

At 612, the user 602 draws or inhales on the vaporizer device 100, which detects the draw or inhale. At 614, in response to the detection of the draw, the vaporizer device 100 operates to provide vapor to the user 602. For example, the vaporizer device 100 applies energy to the heater 166 to produce heat to create vapor from the vaporizable material for inhalation by the user 602.

At 616, consistent with implementations of the current subject matter, the vaporizer device 100 increments the dose counter if the dose control mode is the series dose control mode. The vaporizer device 100 may store the dose counter value and a timer value for the dose consumption on the tag 164 of the cartridge 150.

At 618, upon consumption of the dose, the vaporizer device 100 outputs feedback. The outputted feedback may be based on the dose counter value. For example, the outputted feedback may signify which dose in the series of doses was consumed.

At 620, the user 602 removes the cartridge 150 from the vaporizer device 100. At 622, at some time later, the user 602 reinserts the cartridge 150 into the vaporizer device 100.

At 624, the vaporizer device 100 reads from the tag 164 the dose control settings stored on the tag 164. This allows for, at 626, the vaporizer device 100 to continue or reapply the dose control settings to the vaporizer device 100. The continuation or reapplication of the dose control settings may be based on time for the dose series control mode. For example, upon removal and reinsertion of the cartridge 150, the dose series may continue where the user 602 left off if the timer value has not elapsed. As an example, if the timer value is set at 60 minutes, and the user removes the cartridge 150 after the second dose in a series of four doses and reinserts the cartridge 150 at a time less than 60 minutes, the dose series may continue with the third dose in the series of four doses. If, however, the user 602 inserts a different cartridge 150, then the dose control settings will defer to those, if any, set for the different cartridge 150.

Following the vaporizer device 100 applying or reapplying the dose control settings at 626, consistent with implementations of the current subject matter the process 600 may continue with the user 602 drawing on the vaporizer device 100 (at 628), the vaporizer device 100 operating to provide vapor to the user 602 (at 630), the vaporizer device 100 incrementing the dose counter, and storing the dose counter value and the timer value for the dose consumption on the tag 164 of the cartridge 150 (at 632), and the vaporizer device 100 outputting the feedback based on the dose control mode and/or the dose counter value (at 634).

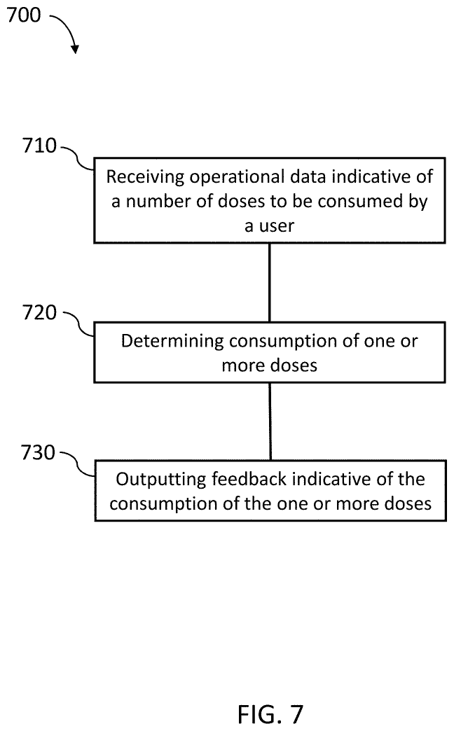

FIG. 7 depicts a chart illustrating a process 700 for operating the vaporizer device 100 consistent with implementations of the current subject matter. In some example embodiments, a controller 128 or other portion of the vaporizer device 100 may perform the process 700.

At 710, the vaporizer device 100 receives operational data that is indicative of a number of doses to be consumed by a user during use of the vaporizer device 100. For example, the vaporizer device 100 may receive from the user device 305 indication of the dose control mode and/or dose control settings selected by the user (e.g., through the app running on the user device 305). Consistent with implementations of the current subject matter, the user may select the continuous dose control mode or the series dose control mode. The dose control settings may include, for example, the timeout period, the timer value, the number of doses to be consumed, and/or the size of the doses to be consumed, where size may be correlated with an amount of total particulate matter and is based on various factors, such as energy. Rather than selecting a particular mode, in some implementations, the user may select various ones of the dose control settings. The vaporizer device 100 may then operate based on the selected dose control settings. In some implementations, the vaporizer device 100 may cause storage of the operational data on the tag 164 of the cartridge 150 in use with the vaporizer device 100.

At 720, following receipt of the operational data indicative of the number of doses to be consumed by the user, the vaporizer device 100 may begin operating (e.g., by applying energy to the heater 166 of the vaporizer device 100 to produce an amount of aerosol correlated with the dose size) and may determine consumption of the dose. Consumption of the dose may be defined as the amount of energy needed to provide the selected dose being applied to the heater 166.

At 730, in response to the determination of the consumption of the dose, the vaporizer device may output feedback indicative of the consumption of the dose. For example, the outputted feedback may include a haptics pulse, an audio indication, a visual indication, or a combination thereof. The outputted feedback may be based on a dose counter value. For example, the outputted feedback may signify which dose in the series of doses was consumed. In some implementations, once the maximum number of doses is consumed, the outputted feedback may indicate, through the outputted feedback, consumption of the desired number of doses. In some implementations, the outputted feedback may correspond to the number of doses consumed (e.g., a first consumed dose may be represented by one haptic pulse, while a second consumed dose may be represented by two haptic pulses).

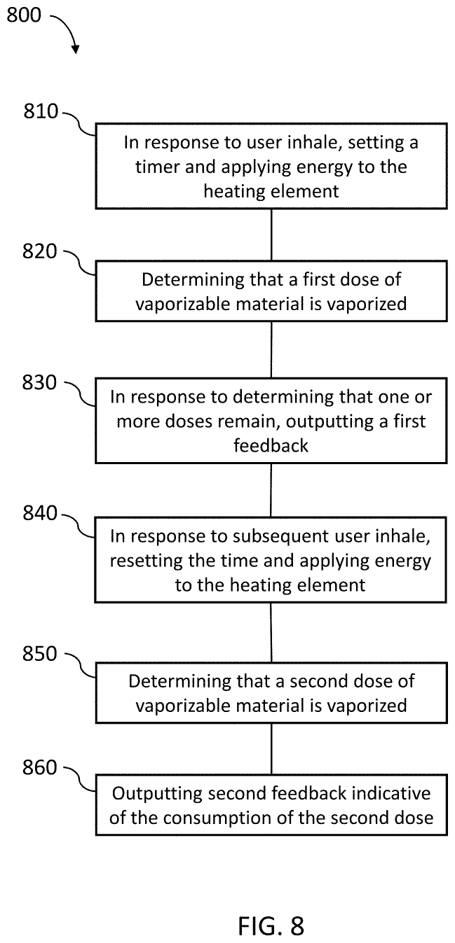

FIG. 8 depicts a chart illustrating a process 800 for operating the vaporizer device 100 consistent with implementations of the current subject matter. In some example embodiments, a controller 128 or other portion of the vaporizer device 100 may perform the process 800.

At 810, in response to a user inhale on the vaporizer device 100, the vaporizer device 100 sets a timer to a predefined timer value and begins applying energy to the heating element (e.g., the heater 166). For example, the heater control circuitry 130 of the vaporizer body 110 may control the heater 166 of the cartridge 150 to generate heat to provide vaporization of the vaporizable material. Moreover, consistent with implementations of the current subject matter, the predefined timer value is indicative of an amount of time between doses when the vaporizer device 100 is operating in the series dose control mode. For example, the predefined timer value defines the amount of time as that which may pass between consumption of doses of the vaporizable material before the series dose control mode ends or is reset.

At 820, a determination is made by the vaporizer device 100 that a first dose of the vaporizable material vaporized by the heating element has been consumed. For example, the dose may be defined as a fixed unit of vapor and may be based on the amount of energy used to produce vapor from the vaporizable material. In some implementations, dose may be based on an amount of energy applied to the heater 166. In some implementations, a partial consumption of a dose may correlate to consumption of the dose if the partial consumption meets or exceeds a predetermined amount. For example, if the predetermined amount is defined as 95%, if 95% of the energy is applied, the dose may be counted as a full dose.

At 830, the vaporizer device 100 determines that one or more doses out of a predefined number of doses remain and responds by outputting a first feedback. Consistent with implementations of the current subject matter, the series dose control mode defines a number of doses to be consumed in the series, and accordingly the vaporizer device 100 tracks the number of doses consumed. Upon the determination of the consumption of the first dose (at 820), the vaporizer device 100 may increment a dose counter, the value of which is compared to the predefined number of doses. In response to determining that additional doses remain, the vaporizer device 100 outputs the first feedback. The first feedback outputted by the vaporizer device 100 signifies to the user that the first dose has been consumed. For example, the dose pulse generated, for example, by the haptics system 144, the animated display of the LEDs 136, and/or the audio and/or visual representation on the user interface of the user device 305 may be outputted. The first feedback may signify a proportion of doses consumed and/or available to be consumed within the current series (e.g., one short pulse for one consumed dose followed by three short pulses to signify three remaining doses, etc.).

At 840, in response to a subsequent user inhale on the vaporizer device 100, the vaporizer device 100 resets or re-initiates the timer to the predefined timer value and again begins applying energy to the heating element (e.g., the heater 166). As the vaporizer device 100 previously determined that doses out of the predefined number of doses remain, the vaporizer device 100 is accordingly responding to a new user inhale by applying energy and tracking the predefined timer value consistent with implementations of the current subject matter.

According to aspects of the current subject matter, the vaporizer device 100 may, in response to the subsequent user inhale, determine that the period of time between the user inhale and subsequent user inhale exceeds the predefined timer value. For example, the vaporizer device 100 may determine that the timer has elapsed, and may respond by clearing the dose counter. As described herein, the timer value may be used to determine if the series of doses should be cleared, for example, if a new series of doses should be started. The clearing of the dose counter, consistent with implementations of the current subject matter, provides for a new series of doses as part of the series dose control mode.

At 850, a determination is made by the vaporizer device 100 that a second dose of the vaporizable material vaporized by the heating element has been consumed. For example, the amount of energy applied by the heating element may be correlated with the dose size. When the amount of energy that correlates with the defined dose size has been reached, the vaporizer device 100 determines that the second dose has been consumed.

At 860, the vaporizer device 100 responds to the determination of the consumption of the second dose by outputting a second feedback. The second feedback outputted by the vaporizer device 100 signifies to the user that another dose has been consumed. For example, the dose pulse generated, for example, by the haptics system 144, the animated display of the LEDs 136, and/or the audio and/or visual representation on the user interface of the user device 305 may be outputted. The second feedback may signify a proportion of doses consumed and/or available to be consumed within the current series. The second feedback may be an end of series feedback, outputted by the vaporizer device 100 when the series is completed (e.g., when the set limited number of doses to be consumed is reached).

In response to the determination of the consumption of the second dose, the vaporizer device may determine that the predetermined number of doses have been consumed. In response to such a determination, a timeout period may be entered. During the timeout period, the heating element (e.g., the heater 166) does not respond to activation commands, such as user puffs or other commands or actions that may be used to start operation of the vaporizer device 100.

Consistent with some implementations of the current subject matter, an override command may be received by the vaporizer device 100 during the timeout period. This determination may be in response to the user inhaling on the vaporizer device 100 and a determination that the timeout period is not over. The timeout period may, consistent with implementations of the current subject matter, be overridden by the user via the app or by a predefined action with respect to the vaporizer device 100. If the timeout period is overridden, the timer may be reset and a new series of doses may start. Based on the vaporizer device 100 having been in the timeout period, the vaporizer device 100 may clear the dose counter.

According to aspects of the current subject matter, the vaporizer device 100 may provide data relating to the dose control settings and the dose control mode to the tag 164 of the cartridge 150 to associate the dose control mode and the dose control settings with the cartridge 150. By storing the dose control mode and the dose control settings, including the predefined timer value, the amount of energy applied to the heating element, the predefined number of dose, and the dose counter value on the tag 164, the dose control mode and the dose control settings are able to be retrieved for subsequent uses. For example, if the user removes the cartridge 150 from the vaporizer body 110 after completion of one or more doses in a series of doses, and then reinserts the cartridge 150 prior to the timer elapsing, the vaporizer device 100 is able to read from the tag 164 the number of doses completed and/or remaining. Thus the vaporizer device 100 is able to resume the series of doses.

Aspects of the current subject matter thus provide for vaporizer device configurations related to dose consumption. For example, aspects of the current subject matter relate to configuring a vaporizer device with respect to providing feedback to a user with respect to dose consumption of one or more vaporizable materials being vaporized and inhaled by a user of a vaporizer device, thus enabling a user to be better informed and/or to monitor dose consumption.

In some examples, the vaporizable material may include a viscous liquid such as, for example a cannabis oil. In some variations, the cannabis oil comprises between 0.3% and 100% cannabis oil extract. The viscous oil may include a carrier for improving vapor formation, such as, for example, propylene glycol, glycerol, medium chain triglycerides (MCT) including lauric acid, capric acid, caprylic acid, caproic acid, etc., at between 0.01% and 25% (e.g., between 0.1% and 22%, between 1% and 20%, between 1% and 15%, and/or the like). In some variations the vapor-forming carrier is 1,3-Propanediol. A cannabis oil may include a cannabinoid or cannabinoids (natural and/or synthetic), and/or a terpene or terpenes derived from organic materials such as for example fruits and flowers. For example, any of the vaporizable materials described herein may include one or more (e.g., a mixture of) cannabinoid including one or more of: CBG (Cannabigerol), CBC (Cannabichromene), CBL (Cannabicyclol), CBV (Cannabivarin), THCV (Tetrahydrocannabivarin), CBDV (Cannabidivarin), CBCV (Cannabichromevarin), CBGV (Cannabigerovarin), CBGM (Cannabigerol Monomethyl Ether), Tetrahydrocannabinol, Cannabidiol (CBD), Cannabinol (CBN), Tetrahydrocannabinolic Acid (THCA), Cannabidioloc Acid (CBDA), Tetrahydrocannabivarinic Acid (THCVA), one or more Endocannabinoids (e.g., anandamide, 2-Arachidonoylglycerol, 2-Arachidonyl glyceryl ether, N-Arachidonoyl dopamine, Virodhamine, Lysophosphatidylinositol), and/or a synthetic cannabinoids such as, for example, one or more of: JWH-018, JWH-073, CP-55940, Dimethylheptylpyran, HU-210, HU-331, SR144528, WIN 55,212-2, JWH-133, Levonantradol (Nantrodolum), and AM-2201. The oil vaporization material may include one or more terpene, such as, for example, Hemiterpenes, Monoterpenes (e.g., geraniol, terpineol, limonene, myrcene, linalool, pinene, Iridoids), Sesquiterpenes (e.g., humulene, farnesenes, farnesol), Diterpenes (e.g., cafestol, kahweol, cembrene and taxadiene), Sesterterpenes, (e.g., geranylfarnesol), Triterpenes (e.g., squalene), Sesquarterpenes (e.g, ferrugicadiol and tetraprenylcurcumene), Tetraterpenes (lycopene, gamma-carotene, alpha- and beta-carotenes), Polyterpenes, and Norisoprenoids. For example, an oil vaporization material as described herein may include between 0.3-100% cannabinoids (e.g., 0.5-98%, 10-95%, 20-92%, 30-90%, 40-80%, 50-75%, 60-80%, etc.), 0-40% terpenes (e.g., 1-30%, 10-30%, 10-20%, etc.), and 0-25% carrier (e.g., medium chain triglycerides (MCT)).

In any of the oil vaporizable materials described herein (including in particular, the cannabinoid-based vaporizable materials), the viscosity may be within a predetermined range. At room temperature of about 23° C., the range may be between about 30 cP (centipoise) and about 200 kcP (kilocentipoise). Alternatively, the range may be between about 30 cP and about 115 kcP. Alternatively, the range may be between about 40 cP and about 113 kcP. Alternatively, the range may be between about 50 cP and about 100 kcP. Alternatively, the range may be between about 75 cP and about 75 kcP. Alternatively, the range may be between about 100 cP and about 50 kcP. Alternatively, the range may be between about 125 cP and about 25 kcP. Outside of these ranges, the vaporizable material may fail in some instances to wick appropriately to form a vapor as described herein. In particular, it is typically desired that the oil may be made sufficiently thin to both permit wicking at a rate that is useful with the apparatuses described herein, while also limiting leaking. For example, viscosities below that of about 30 cP at room temperature might result in problems with leaking, and in some instances viscosities below that of about 100 cP at room temperature might result in problems with leaking.

Although the disclosure, including the figures, described herein may described and/or exemplify these different variations separately, it should be understood that all or some, or components of them, may be combined.

Although various illustrative embodiments are described above, any of a number of changes may be made to various embodiments. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the claims.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. References to a structure or feature that is disposed “adjacent” another feature may have portions that overlap or underlie the adjacent feature.

Terminology used herein is for the purpose of describing particular embodiments only and is not intended to be limiting. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as, for example, “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings provided herein.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” “or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, are possible.

In the descriptions above and in the claims, phrases such as, for example, “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method, comprising:
responsive to a user inhale on a vaporizer device, initiating a timer to a predefined timer value and applying energy to a heating element of the vaporizer device;
determining consumption of a first dose of vaporizable material vaporized by the heating element, the determination of the consumption of the first dose based at least on an amount of the energy applied to the heating element;
outputting, in response to a determination by the vaporizer device that one or more doses out of a predefined number of doses remain, a first feedback;
responsive to a subsequent user inhale on the vaporizer device, re-initiating the timer to the predefined timer value and applying energy to the heating element;
determining consumption of a second dose of vaporizable material vaporized by the heating element, the determination of the consumption of the second dose based at least on the amount of the energy applied to the heating element; and
outputting a second feedback.

2. The method of claim 1, further comprising:
incrementing, in response to the determination of the consumption of the first dose, a dose counter, wherein a value of the dose counter reflects a number of doses consumed.

3. The method of claim 2, further comprising:
responsive to the subsequent user inhale, determining that the period of time between the user inhale and subsequent user inhale exceeds the predefined timer value; and
clearing the dose counter.

4. The method of claim 1, further comprising:
determining, in response to the determination of the consumption of the second dose, that the predetermined number of doses are consumed; and
entering, in response to the determination that the predetermined number of doses are consumed, a timeout period during which the heating element does not respond to activation commands.

5. The method of claim 4, further comprising:
receiving an override command during the timeout period; and
responsive to the override command, re-initiating the timer to the predefined timer value and applying energy to the heating element.

6. The method of claim 4, wherein the second feedback is indicative of the predetermined number of doses being consumed.

7. The method of claim 1, wherein the first feedback is representative of the consumption of the first dose and the second feedback is representative of the consumption of the second dose.

8. The method of claim 1, wherein the determination of the consumption of the first dose is based on the amount of the energy applied to the heating element equal to or exceeding a predetermined value, the predetermined value indicative of at least a partial consumption of the vaporizable material.

9. The method of claim 1, further comprising:
causing storage of one or more of the predefined timer value, the amount of the energy applied to the heating element, and the predefined number of doses on a data tag of a cartridge in use with the vaporizer device.

10. A vaporizer device, comprising:
at least one data processor; and
at least one memory storing instructions which, when executed by the at least one data processor, cause the vaporizer device to at least:
responsive to a user inhale on the vaporizer device, initiate a timer to a predefined timer value and apply energy to a heating element of the vaporizer device;
determine consumption of a first dose of vaporizable material vaporized by the heating element, the determination of the consumption of the first dose based at least on an amount of the energy applied to the heating element;
output, in response to a determination that one or more doses out of a predefined number of doses remain, a first feedback;
responsive to a subsequent user inhale on the vaporizer device, re-initiate the timer to the predefined timer value and apply energy to the heating element;
determine consumption of a second dose of vaporizable material vaporized by the heating element, the determination of the consumption of the second dose based at least on the amount of the energy applied to the heating element; and
output a second feedback.

11. The vaporizer device of claim 10, wherein the vaporizer device is further caused to at least:
increment, in response to the determination of the consumption of the first dose, a dose counter, wherein a value of the dose counter reflects a number of doses consumed.

12. The vaporizer device of claim 11, wherein the vaporizer device is further caused to at least:
responsive to the subsequent user inhale, determine that the period of time between the user inhale and subsequent user inhale exceeds the predefined timer value; and
clear the dose counter.

13. The vaporizer device of claim 10, wherein the vaporizer device is further caused to at least:
determine, in response to the determination of the consumption of the second dose, that the predetermined number of doses are consumed; and
enter, in response to the determination that the predetermined number of doses are consumed, a timeout period during which the heating element does not respond to activation commands.

14. The vaporizer device of claim 13, wherein the vaporizer device is further caused to at least:
receive an override command during the timeout period; and
responsive to the override command, re-initiate the timer to the predefined timer value and apply energy to the heating element.

15. The vaporizer device of claim 13, wherein the second feedback is indicative of the predetermined number of doses being consumed.

16. The vaporizer device of claim 10, wherein the first feedback is representative of the consumption of the first dose and the second feedback is representative of the consumption of the second dose.

17. The vaporizer device of claim 10, wherein the determination of the consumption of the first dose is based on the amount of the energy applied to the heating element equal to or exceeding a predetermined value, the predetermined value indicative of at least a partial consumption of the vaporizable material.

18. The vaporizer device of claim 10, wherein the vaporizer device is further caused to at least:
cause storage of one or more of the predefined timer value, the amount of the energy applied to the heating element, and the predefined number of doses on a data tag of a cartridge in use with the vaporizer device.

19. A non-transitory computer readable medium storing instructions, which when executed by at least one data processor, result in operations comprising:
responsive to a user inhale on a vaporizer device, initiating a timer to a predefined timer value and applying energy to a heating element of the vaporizer device;
determining consumption of a first dose of vaporizable material vaporized by the heating element, the determination of the consumption of the first dose based at least on an amount of the energy applied to the heating element;
outputting, in response to a determination by the vaporizer device that one or more doses out of a predefined number of doses remain, a first feedback;
responsive to a subsequent user inhale on the vaporizer device, re-initiating the timer to the predefined timer value and applying energy to the heating element;
determining consumption of a second dose of vaporizable material vaporized by the heating element, the determination of the consumption of the second dose based at least on the amount of the energy applied to the heating element; and
outputting a second feedback.

20. The non-transitory computer readable medium of claim 19, the operations further comprising:
incrementing, in response to the determination of the consumption of the first dose, a dose counter, wherein a value of the dose counter reflects a number of doses consumed; and
responsive to the subsequent user inhale, determining that the period of time between the user inhale and subsequent user inhale exceeds the predefined timer value; and
clearing the dose counter.
